(19) United States
(12) Patent Application Publication
(10) Pub. No.: US 2010/0076011 A1
(43) Pub. Date: Mar. 25, 2010

(54) Enantiomer-Pure Hexahydro-Pyrrolocyclopenta-Pyridine Derivatives

(76) Inventors: Dieter BINDER, Wien (AT); Eva BINDER, Wien (AT); Michael PYERIN, Brunn am Gebirge (AT)

Correspondence Address: FULBRIGHT & JAWORSKI L.L.P., 600 CONGRESS AVE., SUITE 2400, AUSTIN, TX, 78701, US

(21) Appl. No.: 11/568,445

(22) PCT Filed: Apr. 28, 2005
(86) PCT No.: PCT/EP2005/051921

§ 371 Date: Jun. 29, 2007

(30) Foreign Application Priority Data
Apr. 29, 2004 (AT) A 746/2004
Apr. 29, 2004 (AT) A 747/2004

(51) Int. Cl. A61K 31/437; C07D 471/02; A61P 25/00

(52) U.S. Cl. 514/292; 546/84

(57) ABSTRACT

The invention relates to the novel therapeutically valuable enantiomer-pure [3aα,8bα]-1,2,3a,4,8b-hexahydropyrrolocyclopentapyridine derivatives of the general formula (I), wherein Z is a single bond or CH₂, R1 represents hydrogen or a straight-chain or branched, optionally unsaturated lower alkyl group which may be perfluorated, R2 and R3 independently represent hydrogen, a straight-chain or branched, optionally unsaturated lower alkyl group which may be perfluorated, lower alkoxy, lower alkylthio or halogen, and X and Y alternatively represent CH or N. The invention also relates to the pharmaceutical salts thereof, to a method for their production and to their use.

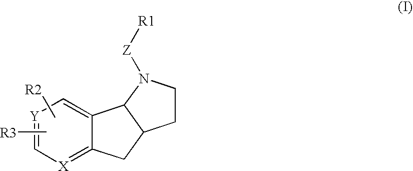

The invention relates to novel therapeutically valuable enantiomerically pure [3aα,8bα]-1,2,3,3a,4,8b-hexahydropyrrolocyclopentapyridine derivatives of the general formula

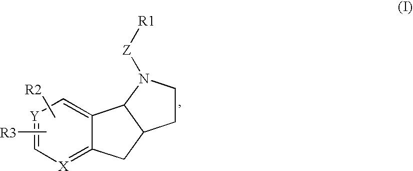

wherein
Z is a single bond or CH₂.
R1 is hydrogen or a straight-chain or branched, optionally unsaturated lower alkyl residue which may also be perfluorated,
R2 and R3 independently represent hydrogen, a straight-chain or branched, optionally unsaturated lower alkyl residue, which may also be perfluorated, lower alkoxy, lower alkylthio or halogen,
X and Y alternately represent CH or N,
   as well as the pharmaceutically usable salts thereof.

The enantiomerically pure compounds and preparations, respectively, according to the present invention have surprisingly positive properties as compared to the enantiomer mixtures and racemates, respectively, of these compounds, primarily with a view to their biological activity, in particular in the CNS range. It has been shown that the respective enantiomers have highly different activities, as will also be demonstrated in Example 9.

Moreover, the present invention relates to a method for producing compounds of the general formula (I), which is characterized in that a compound of the general formula

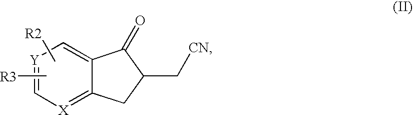

wherein R2, R3, X and Y are as defined above, is reductively converted into the compound of the general formula (I), wherein Z=single bond and R1=hydrogen, the latter compound optionally is reacted with enantiomerically pure 1-phenylethylisocyanate to give the compound of the general formula

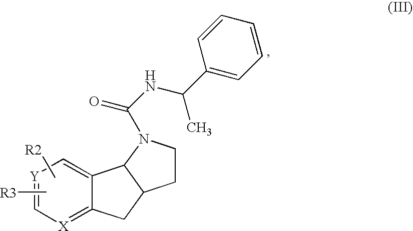

the less readily soluble diastereomer is recovered from the thus-obtained diastereomer mixture by crystallization, and the diastereomerically pure compound of the general formula (III) thus obtained is cleaved under suitable conditions to give the enantiomerically pure compound of the general formula (I), wherein Z=a single bond and R1=hydrogen, the latter compound optionally is reacted under alkylating conditions to compounds of the general formula (I), wherein Z═CH₂, and the compound of the general formula (I) optionally is converted into its pharmaceutically usable salts.

The above-used term “lower alkyl” means a straight-chain or branched alkyl residue having 1-4 carbon atoms, e.g. methyl, ethyl, n- and i-propyl, n-, i- and t-butyl.

The above-used term “lower alkoxy” means a straight-chain or branched alkoxy residue having 1-4 carbon atoms, e.g. methoxy, ethoxy, n- and i-propoxy, n-, i- and t-butoxy.

The above-used term “lower alkylthio” means a straight-chain or branched alkylthio residue having 1-4 carbon atoms, e.g methylthio, ethylthio, n- and i-propylthio, n-, i- and t-butylthio.

The above-used term “halogen” means fluorine, chlorine, bromine or iodine.

The reactions according to the invention are at best carried out in that the compound of the general formula (II) is dissolved in a polar solvent, such as, e.g., acetic acid ethyl ester, dioxane, ethanol or methanol, admixed with 1-5 equivalents of a suitable catalyst, such as, e.g., W2 Raney nickel or Raney cobalt and the like, and hyrogenated at 40 to 70° C. up to the stoichiometric hydrogen uptake.

For enantiomer separation, the resultant racemic compound of the general formula (I) thus obtained, wherein Z=single bond and R1=hydrogen, can be reacted in an inert solvent, such as, e.g., tetrahydrofuran, dioxane or acetone, with 1 equivalent (+) or (−) 1-phenylethyl isocyanate to give a compound of the general formula (III), and from the diastereomer mixture thus obtained, the less readily soluble diastereomer can be recovered by crystallization. For the purpose of cleavage, the thus obtained diastereomerically pure compound of the general formula (III) is dissolved in a high-boiling alcohol, such as, e.g., propanol, butanol, pentanol, glycol etc. or the aqueous mixtures thereof and heated to boiling with 5-20 equivalents of a base, such as sodium propanolate, -butanolate, -pentanolate or sodium hydroxide for 1-24 hours.

The enantiomerically pure compound of the general formula (I) thus obtained, wherein Z=a single bond and R1=hydrogen, optionally is dissolved for the purpose of alkylation in an inert solvent, such as, e.g., tetrahydrofuran, dioxane, acetonitrile or dimethyl formamide etc., with 1-20 equivalents of the compound of the formula

R1-CHO (IV),

wherein R1 is as defined above, and admixed with 1.5-4 equivalents of a reducing agent, such as, e.g., sodium cyanoborohydride or the like, and reacted at −20° C. to 100° C. between 1 and 24 hours.

The compounds of the general formula (I) obtained in this reaction are basic compounds and can be converted into their pharmaceutically compatible salts in conventional manner with inorganic or organic acids. Salt formation may, e.g., be carried out by dissolving the compounds of the formula (I) in a suitable solvent, e.g. water, a lower aliphatic alcohol, THF, dioxane, benzene, diethyl ether, DMF or DMSO, admixing an equivalent amount of the desired acid, providing for good mixing, and removing the solvent in vacuum after salt formation has been completed. Optionally, the salts may be recrystallized after having been isolated.

Pharmaceutically usable salts are those of strong inorganic acids, such as, e.g., hydrochloric acid, hydrobromic acid, sulfuric acid etc., yet also those of organic acids, such as, e.g., fumaric acid, citric acid, sebacic acid etc.

The compounds of the general formula (II), wherein X═N and Y═CH, may, e.g., be obtained according to Scheme 1 (analogous to E. Schröder M. Lehrmann and I. Böttcher, Eur. J. Med. Chem. 1979, 14(4), 309-15, J. Hurst and D. G. Wibberly, J. Chem. Soc. 1962, 119). Accordingly, the optionally R2 and R3-substituted 2-methyl-3-nicotinic acid ester of the general formula (V), wherein R4 represents a lower alkyl residue, is radically brominated with 1.05 equiv. of NBS (N-bromosuccinimide) in boiling carbon tetrachloride, and the crude product is substituted with 1.0 equiv. of sodium malonate in N,N-dimethylformamide. The triester of the general formula (VI) obtained, wherein R5 represents a lower alkyl residue, is subjected to a Dieckmann ester condensation in boiling tetrahydrofuran under the action of 1.05 equiv. of sodium hydride, and by quenching with aqueous ammonium chloride solution is monosaponified to the β-keto ester (VII) and decarboxylated. After deprotonation with sodium hydride and alkylation with iodoacetonitrile in N,N-dimethylformamide, saponification is effected by heating in 2N hydrochloric acid to give the compound of the general formula (II) and decarboxylation is carried out.

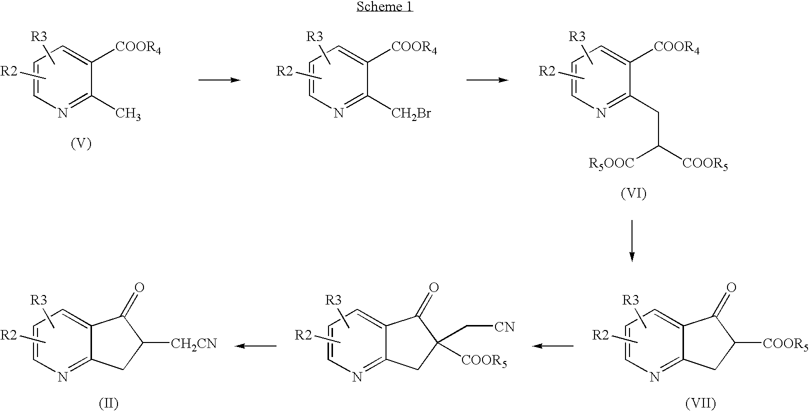

The compounds of the general formula (II), wherein X═CH and Y═N, may for instance be obtained according to Scheme 2, starting from the—optionally R2 and R3-substituted—nicotinic acid ester of the general formula (VIII), wherein R4 represents a lower alkyl residue. The latter is activated with 1.0 equiv. of chloroformic acid ester and reacted with the zinc-copper organyl of a 3-iodopropionic acid ester in tetrahydrofuran to give 1,4-dihydropyridine which is oxidized with sulfur in boiling xylene to the pyridine of the general formula (IX), wherein R5 represents a lower alkyl residue (analogous to M. J. Shiao, W. L. Chia, C. J. Peng and C. C. Shen, J. Org. Chem. 1933, 58, 3162-4). This is cyclized in a Dieckmann ester condensation in boiling methanol under the action of 1.3 equiv. of sodium methanolate to give the β-keto ester (X) (analogous to D. Binder, Monatshefte für Chemie 1974, 105, 196-202). After deprotonation with sodium hydride and alkylation with iodoacetonitrile in N,N-dimethylformamide, saponification is effected by heating in 2N hydrochloric acid to give the compound of the general formula (II), and decarboxylation is carried out.

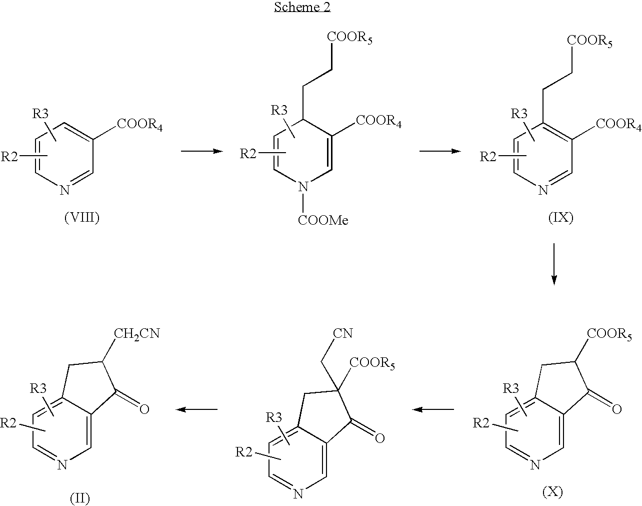

The compounds of the general formulae (IV), (V) and (V) are known from the literature or can be prepared in analogy thereto according to methods known to the person skilled in the art.

The inventive compounds of the general formula (I) and their salts are agonists of central nicotine receptor subtypes and therefore are particularly well suited for the treatment of diseases of the central nervous system, such as, e.g., dementia caused by old age, Alzheimer's Disease, Parkinson Disease, Tourett's Syndrome, dyskinesia, anxiety, depression, panic, psychosis, bulimia, anorexia, and as analgesics, nociceptive agents, neuroprotective agents, for the improvement of perception and attention as well as in the smoke substitution therapy etc.

Due to these pharmacologic properties, the novel compounds, alone or in mixture with other active substances can be used in the form of common galenic preparations as remedies for the treatment of diseases which can be cured or alleviated by the activation of the system of the central nicotin receptor subtypes, or as analgesics, nociceptive agents, neuroprotective agents, for the improvement of perception and attention as well as in the smoke substitution therapy.

The invention further relates to remedies which are used e.g. in the form of pharmaceutical preparations which contain the inventive compounds of the general formula (I) and their salts in mixture with a pharmaceutical organic or inorganic carrier material suitable for oral, enteral, parenteral and topical application, e.g. water, gelatin, gum arabic, lactose, starch, magnesium stearate, talcum, vegetable oils, polyalkylene glycols, Vaseline or the like.

The pharmaceutical preparations may be provided in solid form, e.g. as tablets, film-coated tablets, dragées, suppositories, capsules, microcapsules, or in liquid form, e.g. as solutions, injection solutions, suspensions or emulsions, or in compositions with delayed release of the active substance. Optionally, they are sterilized and/or contain auxiliary agents, such as preservatives, stabilizers or emulsifiers, salts for changing the osmotic pressure, or buffers.

In particular, pharmaceutical preparations can contain the inventive substances in combination with other therapeutically valuable substances. With the latter, the compounds according to the invention can be formulated to combination preparations with the above-indicated auxiliary and/or carrier substances.

The novel compounds may be present in the inventive pharmaceutical compositions in a portion of approximately 1-200 mg per tablet, the balance being a pharmaceutically acceptable filler.

A suitable dose for administering the new compounds is approximately 1-200 mg/kg per day, yet also other doses are possible, depending on the state of the patient to be treated. The novel compounds can be orally administered in several doses.

The following examples explain the invention in more detail, without the latter being restricted thereto:

### EXAMPLE 1

### (+)-[3aα,8bα]-1,2,3,3a,4,8b-Hexahydropyrrolo-[2′,3′:3,4]cyclopenta[1,2-b]pyridine-dihydrochloride

6.68 g (278 mmol) of sodium hydride are admixed at 0° C. with 280 ml of absolute pentanol and stirred for 30 minutes at room temperature. 5.34 g (17.4 mmol) (−)-[1(S),3aα,8bα]-1,2,3,3a,4,8b-hexahydro-N-(1-phenylethyl)-pyrrolo[2′,3′:3,4]cyclopenta[1,2-b]pyridino-1-carboxylic acid amide are added in solid form under N2 counterflow rinsing in one portion, and the reaction mixture is immediately heated to boiling for two hours. The solvent is removed at 60° C./0.1 mbar, and the residue is quickly filtered over 400 g of silica gel with methanol:ammonia=100:2. The solvent is removed, and the crude product is chromatographically purified on a column by gradient elution (400 g of silica gel, dichloromethane:methanol=1:1->methanol methanol:ammonia=100:2). The product obtained is taken up in 20 ml of dichloromethane, dried over sodium sulphate/activated carbon, filtered, and the solvent is distilled off.

Yield: 2.23 g of light-beige crystals (82% of theory)

TLC: methanol:ammonia=100:2; R_{f}=0.5.

With alcoholic hydrochloric acid, the product is converted into its dihydrochloride, crystallized under ethanol, filtered, and digested with acetone. The colourless crystals obtained are highly hygroscopic. [α]_{D}²⁰: +14.4±1.1° (c=0.22/methanol)

Microelementary analysis: RW5 C(%) H(%) N(%) Summation formula: calculated: 51.52 6.05 12.02 C₁₀H₁₄Cl₂N₂ found: 51.52 6.10 11.81

¹H-NMR(D₂O):
δ(ppm)=8.73 (d, 1H, Pcp-H6); 8.69 (d, 1H, Pcp-H8); 7.94 (dd, 1H, Pcp-H7); 5.54 (d, 1H, Pcp-H8b); 3.80-3.20 (m, 5H, Pcp-H2,3a,4A,4B); 2.58-2.36 (m, 1H, Pcp-H3A); 2.09-1.90 (m, 1H, Pcp-H3B)

¹³C-NMR (D₂O):
δ(ppm)=156.2 (s, Pcp-C4a); 142.5 (d, Pcp-C6); 139.7 (d, Pcp-C8); 133.3 (s, Pcp-C8a); 123.1 (d, Pcp-C7); 62.2 (d, Pcp-C8b); 43.5 (t, Pcp-C2); 36.6 (d, Pcp-C3a); 33.1 (t, Pcp-C4); 28.3 (t, Pcp-C3)

### EXAMPLE 2

### (−)-[3aα,8bα]-1,2,3,3a,4,8b-Hexahydropyrrolo-[2′,3′:3,4]cyclopenta[1,2-b]pyridine-dihydrochloride

from 9.10 g (30.0 mmol) (+)-[1(R),3aα,8bα]-1,2,3,3a,4,8b-hexahydro-N-(1-phenylethyl)-pyrrolo-[2′,3′:3,4]cyclopenta[1,2-b]pyridino-1-carboxylic acid amide analogous to Example 1 (72% of theory, colorless crystals).

[α]_{D}²⁰: −13.9±1.0° (c=0.24/methanol)

Microelementary analysis: RW7 C(%) H(%) N(%)
Summation formula: calculated: 51.16 6.09 11.93 C₁₀H₁₄Cl₂N₂*0.09 H₂O found: 51.22 6.24 11.80

¹H-NMR (D₂O):
δ(ppm)=8.73 (d, 1H, Pcp-H6); 8.69 (d, 1H, Pcp-H8); 7.94 (dd, 1H, Pcp-H7); 5.54 (d, 1H, Pcp-H8b); 3.80-3.20 (m, 5H, Pcp-H2,3a,4A,4B); 2.58-2.36 (m, 1H, Pcp-H3A); 2.09-1.90 (m, 1H, Pcp-H3B)

¹³C-NMR (D₂O):
δ(ppm)=156.2 (s, Pcp-C4a); 142.5 (d, Pcp-C6); 139.7 (d, Pcp-C8); 133.3 (s, Pcp-C8a); 123.1 (d, Pcp-C7); 62.2 (d, Pcp-C8b); 43.5 (t, Pcp-C2); 36.6 (d, Pcp-C3a); 33.1 (t, Pcp-C4); 28.3 (t, Pcp-C3)

The starting material can be prepared as follows:

### 2-[2,2-Bis-(methoxycarbonyl)]-ethyl-3-pyridino-carboxylic acid methyl ester

219.0 g (1.45 mol) of 2-methyl-3-pyridino carboxylic acid methyl ester in 3.5 l of absolute tetrachloromethane are heated to boiling over night with 271.0 g (1.52 mol) of N-bromosuccinimide and 13.0 g of dibenzoyl peroxide.

The solid is filtered off, the solvent is removed, the residue is dissolved in 250 ml of absolute dimethyl formamide and added dropwise at 10° C. to a suspension of 223.3 g (1.45 mol) of sodium dimethyl malonate in 1.2 l of absolute dimethyl formamide and stirred at room temperature for 18 hours.

The solvent is removed under high vacuum, the residue is partitioned between 2.5 l of water and 1.5 l of diethyl ether, and the aqueous phase is extracted four times with 800 ml of diethyl ether each. The combined organic phases are washed with 200 ml of water, dried over sodium sulfate/activated carbon, filtered, the solvent is removed, and the residue is digested with 200 ml of diethyl ether.

Yield: 138.5 g of colorless crystals (34% of theory)

TLC: acetic acid ethyl ester; R_{f}=0.6

M.p.: 59-62° C. (diethyl ether, dig.)

Microelementary analysis: HK29 C(%) H(%) N(%)
Summation formula: calculated: 55.51 5.38 4.98 C₁₀H₁₄Cl₂N₂ found: 55.71 5.26 4.92

¹H-NMR(CDCl₃):
δ(ppm)=8.55 (dd, 1H, Py-H6); 8.18 (dd, 1H, Py-H4); 7.20 (dd, 1H, Py-H5); 4.22 (t, 1H, CH); 3.90 (s, 3H₂OCH₃); 3.80 (d, 2H, CH₂); 3.70 (s, 6H₂OCH₃)

¹³C-NMR(CDCl₃):
δ(ppm)=169.7 (s, 2C,COOCH₂); 166.2 (s, COOCH₃); 158.4 (s, Py-C2); 151.3 (d, Py-C6); 138.3 (d, Py-C4); 125.0 (s, Py-C3); 121.2 (d, Py-C5); 52.2 (q, 2C, OCH₃); 52.1 (q, OCH₃); 49.6 (d, CH); 34.9 (t, CH₂)

### 6,7-Dihydro-5-oxo-5H-1-pyridino-6-carboxylic acid methyl ester

To a suspension of 8.40 g (350 mmol) of sodium hydride in 800 ml of boiling absolute tetrahydrofuran, there are slowly added 93.5 g (332 mmol) of 2-[2,2-bis(methoxycarbonyl)]-ethyl-3-pyridinocarboxylic acid methyl ester in 600 ml of absolute hot tetrahydrofuran. The reaction solution is refluxed until gas development has ceased, with the product precipitating.

The cooled suspension is poured onto 1.5 l of saturated ammonium chloride solution, stirred for 30 minutes, and the precipitate is filtered off. The solid is washed three times with 250 ml of water, digested once with 250 ml of methanol and dried over phosphoropentoxide at 70° C./20 mbar.

Yield: 55.4 g of colorless crystals (87% of theory)

TLC: acetic acid ethyl ester; R_{f}=0.4

M.p.: 92-96° C. (acetic acid ethyl ester)

Microelementary analysis: C(%) H(%) N(%)
Summation formula: calculated: 62.82 4.74 7.32 C₁₀H₉NO₃ found: 63.02 4.79 7.32

¹H-NMR(CDCl₃):
Keto form: δ(ppm)=8.82 (dd, 1H, Pn-H2); 8.02 (dd, 1H, Pn-H4); 7.35 (dd, 1H, Pn-H3); 3.79 (dd, 1H, Pn-H6); 3.78 (s, 3H₂OCH₃); 3.72-3.40 (m, 2H, Pn-H7A,B)
Enol form: δ(ppm)=8.58 (dd, 1H, Pn-H2); 7.87 (dd, 1H, Pn-H4); 7.27 (dd, 1H, Pn-H3); 3.86 (s, 3H₂OCH₃); 3.61 (s, 2H, Pn-H7)

¹³C-NMR(CDCl₃):
Keto form: δ(ppm)=197.6 (s, Pn-C5); 172.7 (s, COOCH₃); 168.8 (s, Pn-C7a); 156.3 (d, Pn-C2); 132.7 (d, Pn-C4); 128.5 (s, Pn-C4a); 122.8 (d, Pn-C3); 52.8 (d, Pn-C6); 52.7 (q, OCH₃); 32.9 (t, Pn-C7)
Enol form: δ(ppm)=169.3 (s, COOCH₃); 167.1 (s, Pn-C5)*; 163.5 (s, Pn-C7a)*; 149.8 (d, Pn-C2); 131.9 (s, Pn-C4a)*; 128.1 (d, Pn-C4); 121.7 (d, Pn-C3); 102.0 (s, Pn-C6); 51.3 (q, OCH₃); 34.8 (t, Pn-C7)

### 6-(Cyanomethyl)-6,7-dihydro-5-oxo-5H-1-pyridino-6-carboxylic acid methyl ester

To a suspension of 7.66 g (319 mmol) of sodium hydride in 400 ml of absolute dimethyl formamide, there are added 55.4 g (290 mmol) of 6.7-dihydro-5-oxo-5H-1-pyridino-6-carboxylic acid methyl ester at 5° C. and it is stirred until gas development has ceased. After the addition of 55.7 g (333 mmol) of iodoacetonitrile in 200 ml of absolute dimethylformamide, it is first stirred for 30 minutes at 5° C. and subsequently for 18 hours at room temperature. The pH is adjusted to 5 with glacial acetic acid, dimethyl formamide is removed under fine vacuum, the residue is taken up in 900 ml of water, it is extracted twice with dichloromethane with 600 ml each and three times with 300 ml each. The organic phase is dried over sodium sulfate/activated carbon, filtered, and the solvent is distilled off. After start-spot filtration (1.2 kg of silica gel KG60, acetic acid ethyl ester), the crude product is recrystallized from 200 ml of acetic acid ethyl ester.

Yield: 35.6 g of colorless crystals (53% of theory)

TLC: acetic acid ethyl ester; R_{f}=0.6

M.p.: 99-101° C. (acetic acid ethyl ester)

Microelementary analysis: FG1 C(%) H(%) N(%)
Summation formula: calculated: 62.61 4.38 12.17 C₁₂H₁₀N₂O₃ found: 62.45 4.27 12.16

¹H-NMR(CDCl₃):
δ(ppm)=8.90 (dd, 1H, Pn-H2); 8.09 (dd, 1H, Pn-H4); 7.44 (dd, 1H, Pn-H3); 3.87 (d, 1H, Pn-H7A); 3.71 (s, 3H, OCH₃); 3.42 (d, 1H, Pn-H₇B); 3.20 (d, 1H, CHAHB—CN); 2.98 (d, 1H, CHAHB—CN)

¹³C-NMR(CDCl₃):
δ(ppm)=197.4 (s, Pn-C5); 171.6 (s, COOCH₃); 168.6 (s, Pn-C7a); 157.1 (d, Pn-C2); 133.4 (d, Pn-C4); 127.5 (s, Pn-C4a); 123.4 (d, Pn-C3); 116.0 (s, CN); 56.6 (s, Pn-C6); 53.6 (q, OCH₃); 39.4 (t, Pn-C7); 22.1 (t, CH₂CN)

### 6,7-Dihydro-5-oxo-5H-1-pyridino-6-carboxylic acid nitrile

35.6 g (155 mmol) of 6-(cyanomethyl)-6,7-dihydro-5-oxo-1-pyridino-6-carboxylic acid methyl ester are heated to boiling in 400 ml of 2N hydrochlorid acid for 90 minutes. The reaction solution is adjusted to pH=9 with solid sodium carbonate, extracted twice with 200 ml of dichloromethane each and three times with 100 ml each. The organic phase is dried over sodium sulfate/activated carbon, filtered, and the solvent is distilled off.

Yield: 24.0 g of colorless crystals (90% of theory)

TLC: acetic acid ethyl ester; R_{f}=0.5

M.p.: 95-97° C. (dichloromethane)

Microelementary analysis: FG2 C(%) H(%) N(%)
Summation formula: calculated: 69.76 4.68 16.27 C₁₀H₈N₂O found: 69.54 4.76 16.20

¹H-NMR(CDCl₃):
δ(ppm)=8.87 (dd, 1H, Pn-H2); 8.06 (dd, 1H, Pn-H4); 7.37 (dd, 1H, Pn-H3); 3.66 (dd, 1H, Pn-H7A); 3.22-2.92 (m, 3H, Pn-H₇B, Pn-H6, CHAHB—CN); 2.73 (dd, 1H, CHAHB—CN)

¹³C-NMR(CDCl₃):
δ(ppm)=202.2 (s, Pn-C5); 171.7 (s, Pn-C7a); 156.5 (d, Pn-C2); 132.4 (d, Pn-C4); 128.9 (s, Pn-C4a); 123.0 (d, Pn-C3); 117.2 (s, CN); 42.7 (d, Pn-C6); 34.5 (t, Pn-C7); 18.3 (t, CH₂CN)

### (±)-[3aα,8bα]-1,2,3,3a,4,8b-Hexahydropyrrolo-[2′,3′:3,4]cyclopenta[1,2-b]pyridine

4.0 g (23.23 mmol) of 6,7-dihydro-5-oxo-5H-1-pyridino-6-acetic acid nitrile are dissolved in 80 ml of absolute methanol, stirred with activated carbon and filtered. The solution is admixed with 16 g of Raney cobalt catalyst and hydrogenated in a Parr apparatus at 50° C. and 90 psi hydrogen pressure until the end of the theoretical hydrogen uptake. The catalyst is filtered off via Hyflo, the solvent of the filtrate is distilled off, and the residue is purified by start spot filtration over 400 g of silica gel with methanol:ammonia=100:2. The solvent is distilled off, the residue is taken up in dichloromethane, dried over sodium sulfate/activated carbon, filtered, and the solvent is removed.

Yield: 2.61 g of a colorless oil (70% of theory)

TLC: methanol:ammonia=100:2; R_{f}=0.5

¹H-NMR(CDCl₃):
δ(ppm)=8.42 (dd, 1H, Pcp-H6); 7.62 (dd, 1H, Pcp-H8); 7.09 (dd, 1H, Pcp-H7); 4.74 (d, 1H, Pcp-H8b); 3.30 (dd, 1H, Pcp-H4A); 3.12-2.61 (m, 4H, Pcp-H4B,2,3a); 2.15-1.94 (m, 1H, Pcp-H3A); 1.68-1.49 (m, 1H, Pcp-H3B)

¹³C-NMR(CDCl₃):
δ(ppm)=164.0 (s, Pcp-C4a); 149.3 (d, Pcp-C6); 137.4 (s, Pcp-C8a); 133.1 (d, Pcp-C8); 121.7 (d, Pcp-C7); 66.5 (d, Pcp-C8b); 46.6 (t, Pcp-C2); 40.2 (d, Pcp-C3a); 39.3 (t, Pcp-C4); 35.7 (t, Pcp-C3)

### (−)-[1(S)3aα,8bα]-1,2,3,3a,4,8b-Hexahydro-N-(1-phenylethyl)-pyrrolo[2′,3′:3,4]cyclopenta[1,2-b]pyridino-1-carboxylic acid amide

2.50 g (15.6 mmol) (±)-[3aα,8bα]-1,2,3,3a,4,8b-hexahydropyrrolo[2′,3′:3,4]cyclopenta[1,2-b]pyridine in 30 ml of absolute acetone are admixed with 2.30 g (15.6 mmol) of (S)-(−)-1-phenylethyl-isocyanate in 25 ml of absolute acetone, stirred at room temperature for 60 minutes and put into the refrigerator over night for completion of crystallization. The crystals are filtered off and digested with cold absolute acetone.

Yield: 2.04 g of colorless crystals (85% of theory)

TLC: dichloromethane:methanol=95:5; R_{f}=0.5

M.p.: 187-189° C. (acetone)
[α]_{D}²⁰: −229.5±0.5° (c=1.00/dichloromethane)

Microelementary analysis: GD28 C(%) H(%) N(%)
Summation formula: calculated: 74.24 6.89 13.67 C₁₉H₂₁N₃O found: 74.24 7.10 13.53

¹H-NMR(CDCl₃):
δ(ppm)=8.39 (d, 1H, Pcp-H6); 8.03 (d, 1H, Pcp-H8); 7.34-7.21 (m, 5H, Bz-H2-4); 7.06 (dd, 1H, Pcp-H7); 5.36 (d, 1H, Pcp-H8b); 5.05 (m, 1H, CH); 4.63 (d, 1H; NH); 3.36-3.29 (m, 2H, Pcp-H2); 3.18 (dd, 1H, Pcp-H4A); 3.10-2.90 (m, 1H, Pcp-H3a); 2.83 (dd, 1H, Pcp-H4B); 2.29-2.14 (m, 1H, Pcp-H3A); 1.79-1.60 (m, 1H, Pcp-H3B); 1.48 (d, 3H, CH₃)

¹³C-NMR(CDCl₃):
δ(ppm)=161.9 (s, Pcp-C4a); 156.3 (s, CO); 149.0 (d, Pcp-C6); 144.4 (s, Bz-C1); 137.3 (s, Pcp-C8a); 135.6 (d, Pcp-C8); 128.4 (d, 2C, Bz-C3); 126.9 (d, Bz-C4); 125.9 (d, 2C, Bz-C2); 121.9 (d, Pcp-C7); 64.5 (d, Pcp-C8b); 49.7 (d, CH); 45.7 (t, Pcp-C2); 39.0 (d, Pcp-C3a); 37.8 (t, Pcp-C4); 31.3 (t, Pcp-C3); 22.5 (q, CH₃)

(+)-[1(R)₃aα,8bα]-1,2,3,3a,4,8b-Hexahydro-N-(1-phenylethyl)-pyrrolo[2′,3′:3,4]cyclopenta[1,2-b]pyridino-1-carboxylic acid amide from 512 g (31.9 mmol) (±)-[3aα,8bα]-1,2,3,3a,4,8b-hexahydropyrrolo[2′,3′:3,4]cyclopenta[1,2-b]pyridine and 4.47 g (30.3 mmol) (R)-(+)-1-phenylethylisocyanate analogous to (−)-[1(S)₃aα,8bα]-1,2,3,3a,4,8b-hexahydro-N-(1-phenylethyl)-pyrrolo[2′,3′:3,4]cyclopenta[1,2-b]pyridino-1-carboxylic acid amide

Yield: 4.19 g of colorless crystals (85% of theory)

TLC: dichloromethane:methanol=95:5; R_{f}=0.5

M.p.: 184-186° C. (acetone)

[α]_{D}²⁰: +230.4±0.5° (c=0.19/dichloromethane)

Microelementary analysis: RW1 C(%) H(%) N(%)
Summation formula: calculated: 74.24 6.89 13.67 C₁₉H₂₁N₃O found: 74.26 7.04 13.56

¹H-NMR(CDCl₃):
δ(ppm)=8.39 (d, 1H, Pcp-H6); 8.03 (d, 1H, Pcp-H8); 7.34-7.21 (m, 5H, Bz-H2-4); 7.06 (dd, 1H, Pcp-H7); 5.36 (d, 1H, Pcp-H8b); 5.05 (m, 1H, CH); 4.63 (d, 1H, NH); 3.36-3.29 (m, 2H, Pcp-H2); 3.18 (dd, 1H, Pcp-H4A); 3.10-2.90 (m, 1H, Pcp-H3A); 2.83 (dd, 1H, Pcp-H4B); 2.29-2.14 (m, 1H, Pcp-H3A); 1.79-1.60 (m, 1H, Pcp-H3B); 1.48 (d, 3H, CH₃)

¹³C-NMR(CDCl₃):
δ(ppm)=161.9 (s, Pcp-C4a); 156.3 (s, CO); 149.0 (d, Pcp-C6); 144.4 (s, Bz-C1); 137.3 (s, Pcp-C8a); 135.6 (d, Pcp-C8); 128.4 (d, 2C, Bz-C3); 126.9 (d, Bz-C4); 125.9 (d, 2C, Bz-C2); 121.9 (d, Pcp-C7); 64.5 (d, Pcp-C8b); 49.7 (d, CH); 45.7 (t, Pcp-C2); 39.0 (d, Pcp-C3a); 37.8 (t, Pcp-C4); 31.3 (t, Pcp-C3); 22.5 (q, CH₃)

### EXAMPLE 3

### (−)-[3aα,8bα]-1,2,3,3a,4,8b-Hexahydro-1-methyl-pyrrolo[2′,3′:3,4]cyclopenta[1,2-b]pyridine-dihydrochloride

1.15 g (7.17 mmol) of [3aα,8bα]-1,2,3,3a,4,8b-hexahydropyrrolo[2′,3′:3,4]cyclopenta[1,2-b]pyridine (Example 1) in 60 ml acetonitrile are admixed with 5.42 ml of 35% formaldehyde solution and subsequently, in portions, with 1.04 g (16.5 mmol) of sodium cyanoborohydride. The reaction mixture is stirred at room temperature for 30 minutes. Now the pH is adjusted to 1 with 2N hydrochloric acid, and it is extracted twice with 30 ml of dichloromethane each. By the addition of 2N caustic soda solution, the aqueous phase is adjusted to a pH >11, and it is extracted six times with 30 ml of dichloromethane each. The organic phase is dried over sodium sulfate/activated carbon, filtered, and the solvent is distilled off.

Yield: 1.13 g of a yellow oil (90% of theory)

TLC: methanol:ammonia=100:2; R_{f}=0.8 methylen-chloride:methanol=10:1; R_{f}=0.5

The product is converted into its dihydrochloride with alcoholic hydrochloric acid, crystallized under ethanol, filtered off and digested with acetone. The colorless crystals obtained are highly hygroscopic.

[α]_{D}²⁰: −29.6±1.0° (c=0.44/methanol)

Microelementary analysis: RW16 C(%) H(%) N(%)
Summation formula: calculated: 48.83 6.93 10.35 C₁₁H₁₆N₂Cl₂*1.30 H₂O found: 48.84 6.79 10.22

¹H-NMR(D₂O):
δ(ppm)=8.78 (d, 1H, Pcp-H6); 8.75 (d, 1H, Pcp-H8); 7.96 (dd, 1H, Pcp-H7); 5.28 (d, 1H, Pcp-H8b); 3.85-3.50 (m, 3H, Pcp-H2,4A); 3.19 (s, 3H, CH₃); 3.50-3.00 (m, 2H, Pcp-H4B,3a); 2.80-2.50 (m, 1H, Pcp-H3A); 2.05-1.80 (m, 1H, Pcp-H3B)

¹³C-NMR (D₂O):
δ(ppm)=161.5 (s, Pcp-C4a); 146.8 (d, Pcp-C6); 145.5 (d, Pcp-C8); 137.2 (s, Pcp-C8a); 128.2 (d, Pcp-C7); 76.7 (d, Pcp-C8b); 59.4 (t, Pcp-C2); 43.1 (d, Pcp-C3a); 41.9 (q, CH₃); 38.4 (t, Pcp-C4); 32.9 (t, Pcp-C3)

### EXAMPLE 4

### (+)-[3aα,8bα]-1,2,3,3a,4,8b-Hexahydro-1-methyl-pyrrolo[2′,3′:3,4]cyclopenta[1,2-b]pyridinedihydrochloride

from 1.13 g (7.17 mmol) of [3aα,8bα]-1,2,3,3a,4,8b-hexahydropyrrolo[2′,3′:3,4]cyclopenta[1,2-b]pyridine (Example 2), 5.42 ml of 35% formaldehyde solution and 1.04 g (16.5 mmol) sodiumcyanoborohydride analogous to Example 3 (86% of theory, colorless crystals).

[α]_{D}²⁰: +27.5±0.5° (c=0.42/methanol)

Microelementary analysis: RW8 C(%) H(%) N(%)
Summation formula: calculated: 48.96 6.92 10.38 C₁₁H₁₆N₂Cl₂*1.26H₂O found: 48.92 6.64 10.48

¹H-M (D₂O):
δ(ppm)=8.78 (d, 1H, Pcp-H6); 8.75 (d, 1H, Pcp-H8); 7.96 (dd, 1H, Pcp-H7); 5.28 (d, 1H, Pcp-H8b); 3.85-3.50 (m, 3H, Pcp-H2,4A); 3.19 (s, 3H, CH₃); 3.50-3.00 (m, 2H, Pcp-H4B,3a); 2.80-2.50 (m, 1H, Pcp-H3A); 2.05-1.80 (m, 1H, Pcp-H3B)

¹³C-NMR (D₂O):
δ(ppm)=161.5 (s, Pcp-C4a); 146.8 (d, Pcp-C6); 145.5 (d, Pcp-C8); 137.2 (s, Pcp-C8a); 128.2 (d, Pcp-C7); 76.7 (d, Pcp-C8b); 59.4 (t, Pcp-C2); 43.1 (d, Pcp-C3a); 41.9 (q, CH₃); 38.4 (t, Pcp-C4); 32.9 (t, Pcp-C3)

### EXAMPLE 5

### (+)-(3aS-(3aα,8bα)]-1,2,3,3a,4,8b-Hexahydropyrrolo[3′,2′:4,5]-cyclopenta[1,2-c]-pyridinodihydrochloride

6.00 g (250 mmol) of sodium hydride are admixed with 240 ml of abs. pentanol at 0° C. and stirred for 30 minutes. 4.80 g (15.6 mmol) of [1S-[1R*(R*),2(R*))]-1,2,3,3a,4,8b-hexahydro-N-(1-phenylethyl)-pyrrolo-[3′2′:4,5]cyclopenta[1,2-c]-pyridino-1-carboxamide are admixed in solid form in one portion, and the reaction mixture is heated to boiling for 2 hours. The solvent is removed at 60° C./0.1 mbar, and the residue is quickly filtered over 500 g of silica gel KG60 (methanol:ammonia=100:2). The crude product is chromatographically purified on a column (250 g of silica gel KG60; methanol:ammonia=100:2). The product obtained is taken up in 20 ml of dichloromethane, dried over sodium sulfate/activated carbon, filtered, and the solvent is distilled off.

Yield: 1.93 g of beige crystals (77% of theory)

TLC: methanol:ammonia=100:2; R_{f}=0.25

The product is converted into its dihydrochloride with alcoholic hydrochloric acid, crystallized under ethanol, filtered off and digested with acetone. The colorless crystals obtained are highly hygroscopic.

[α]_{D}²⁰: +37.9° (c=0.12/methanol)

Microelementary analysis: HA42 C(%) H(%) N(%)
Summation formula: calculated: 50.50 6.15 11.78 C₁₀H₁₄Cl₂N₂*0.26H₂O found: 50.64 6.08 11.55

¹H-NMR(D₂O):
δ(ppm)=8.97 (s, 1H, Pcp-H8); 8.72 (d, 1H, Pcp-H6); 8.03 (d, 1H, Pcp-H5); 5.58 (d, 1H, Pcp-H8b); 3.78-3.40 (m, 3H, Pcp-H3a,4A,B); 3.38-3.18 (m, 2H, Pcp-H2A,B); 2.54-2.30 (m, 1H, Pcp-H3A); 2.04-1.87 (m, 1H, Pcp-H3B)

¹³C-NMR (D₂O):
δ(ppm)=168.8 (s, Pcp-C4a); 143.3 (d, Pcp-C8); 141.1 (d, Pcp-C6); 138.0 (s, Pcp-C8a); 126.1 (d, Pcp-C5); 67.0 (d, Pcp-C8b); 47.8 (t, Pcp-C2); 42.6 (d, Pcp-C3a); 40.2 (t, Pcp-C4); 32.6 (t, Pcp-C3)

### EXAMPLE 6

### (−)-[3aR-(3aα,8bα)]-1,2,3,3a,4,8b-Hexahydropyrrolo[3′,2′:4,5]-cyclopenta[1,2-c]pyridinodihydrochloride

from 1.00 g (3.26 mmol) of [1R-[1R*(R*),2(S*)]]-1,2,3,3a,4,8b-hexahydro-N-(1-phenylethyl)pyrrolo[3′2′:4,5]cyclopenta[1,2-c]-pyridino-1-carboxamide analogous to Example 5 (83% of theory, colorless crystals)

[α]_{D}²⁰: −36.8° (c=0.11/methanol)

Microelementary analysis: HA43 C(%) H(%) N(%)
Summation formula: calculated: 50.58 6.15 11.80 C₁₀H₁₄Cl₂N₂*0.25H₂O found: 50.64 6.04 11.55

¹H-NMR(D₂O):
δ(ppm)=8.97 (s, 1H, Pcp-H8); 8.72 (d, 1H, Pcp-H6); 8.03 (d, 1H, Pcp-H5); 5.58 (d, 1H, Pcp-H8b); 3.78-3.40 (m, 3H, Pcp-H3a,4A,B); 3.38-3.18 (m, 2H, Pcp-H2A,B); 2.54-2.30 (m, 1H, Pcp-H3A); 2.04-1.87 (m, 1H, Pcp-H3B)

¹³C-NMR (D₂O):
δ(ppm)=168.7 (s, Pcp-C4a); 143.3 (d, Pcp-C8); 141.1 (d, Pcp-C6); 137.9 (s, Pcp-C8a); 126.0 (d, Pcp-C5); 67.0 (d, Pcp-C8b); 47.8 (t, Pcp-C2); 42.6 (d, Pcp-C3a); 40.2 (t, Pcp-C4); 32.5 (t, Pcp-C3);

The starting material can be prepared as follows:

### 6-Cyanomethyl-5,6-dihydro-7-oxo-7H-2-pyridino-6-carboxylic acid methyl ester

To a suspension of 4.51 g (188 mmol) of sodium hydride in 500 ml of abs. dimethylformamide, 30.00 g (157 mmol) of 5.6-dihydro-7-oxo-7H-2-pyridino-6-carboxylic acid ester are added in portions at 0° C. The suspension thus forming is stirred at room temperature for 2 hours, 31.40 g (188 mmol) of iodoacetonitrile are added at 0° C., and the mixture is stirred over night at room temperature.

The solvent is removed at 60° C./0.1 mbar, and the residue is partitioned between 500 ml of water and a total of 4 l of diethylether. The combined organic phases are dried over sodium sulfate/activated carbon, filtered, and the extraction agent is removed. The crude product is recrystallized from 250 ml of ethanol.

Yield: 23.50 g of orange crystals (65% of theory)

TLC: EE; R_{f}=0.35

M.p.: 102-103° C. (ethanol)

Microelementary analysis: HA32 C(%) H(%) N(%)
Summation formula: calculated: 62.61 4.48 12.17 C₁₂H₁₀N₂O₃ found: 62.39 4.33 12.14

¹H-NMR(CDCl₃):
δ(ppm)=9.08 (s, 1H, Pn-H1); 8.82 (d, 1H, Pn-H3); 7.53 (dd, 1H, Pn-H4); 3.72 (s, 3H₂OCH₃); 3.83-3.36 (AB, 1H, Pn-H5A); 3.21-2.29 (AB, 1H, CH_{A}H_{B}—CN)

¹³C-NMR(CDCl₃):
δ(ppm)=197.6 (s, C═O); 168.3 (s, COOCH₃); 160.1 (s, Pn-C4a); 154.9 (d, Pn-C1); 147.7 (d, Pn-C3); 129.8 (s, Pn-C7a); 121.7 (d, Pn-C4); 116.1 (s, CN); 56.6 (s, Pn-C6); 53.6 (q, OCH₃); 36.7 (t, Pn-C5); 22.1 (t, CH₂CN)

### 5,6-Dihydro-7-oxo-7H-2-pyridino-6-acetic acid nitrile

11.6 g (50.4 mmol) of 6-cyanomethyl-5,6-dihydro-7-oxo-7H-2-pyridino-6-carboxylic acid methyl ester are heated to boiling in 180 ml of 2N hydrochloric acid for 4 hours, subsequently adjusted to pH=9 with solid sodium hydrogencarbonate and extracted seven times with 100 ml of acetic acid ethyl ester each. The combined organic phases are washed with 100 ml of water, dried over sodium sulfate/activated carbon, filtered, and the solvent is removed. The product is digested several times with diethyl ether.

Yield: 7.20 g of dark-green crystals (83% of theory)

TLC: EE; R_{f}=0.2

M.p.: 94-95° C. (diethyl ether)

Microelementary analysis: HA33 C(%) H(%) N(%)
Summation formula: calculated: 69.25 4.73 16.15 C₁₀H₈N₂O*0.07H₂O found: 69.26 4.86 16.00

¹H-NMR(CDCl₃):
δ(ppm)=9.01 (s, 1H, Pn-H1); 8.75 (d, 1H, Pn-H3); 7.49 (dd, 1H, Pn-H4); 3.72-3.40 (m, 1H, Pn-H6); 3.14-2.89 (m, 3H, Pn-H5A,B, CH_{A}H_{B}—CN); 2.77-2.60 (m, 1H, CH_{A}H_{B}—CN)

¹³C-NMR(CDCl₃):
δ(ppm)=202.2 (s, C═O); 160.0 (s, Pn-C4a); 153.9 (d, Pn-C1); 146.2 (d, Pn-C3); 130.9 (s, Pn-C7a); 121.6 (d, Pn-C4); 117.0 (s, CN); 42.3 (d, Pn-C6); 31.3 (t, Pn-C5); 17.7 (t, CH₂CN)

### (±)-[3aα,8bα]-1,2,3,3a,4,8b-Hexahydropyrrolo-[3′,2′:4,5]-cyclopenta[1,2-c]pyridine

3.45 g (20.0 mmol) of 5,6-dihydro-7-oxo-7H-2-pyridino-6-acetic acid nitrile are dissolved in 110 ml of abs. methanol, stirred with activated carbon, filtered, admixed with 12 g of Raney cobalt as a catalyst, and hydrogenated in a Parr apparatus at 50° C. and 90 psi until the theoretical hydrogen uptake had ceased.

Raney-cobalt is filtered off via Hyflo, the solvent of the filtrate is distilled off, and the residue is chromatographically purified on a column (135 g of silica gel KG60; methanol:ammonia=100:2)

Yield: 2.00 g of brown crystals (62% of theory)

TLC: methanol:ammonia=100:2; R_{f}=0.25.

Microelementary analysis: HA34A C(%) H(%) N(%)
Summation formula: calculated: 74.97 7.55 17.48 C₁₀H₁₂N₂ found: 74.71 7.57 17.39

¹H-NMR(CDCl₃):
δ(ppm)=8.56 (s, 1H, Pcp-H8); 8.38 (d, 1H, Pcp-H6); 7.09 (d, 1H, Pcp-H5); 4.85 (d, 1H, Pcp-H8b); 3.28-3.13 (m, 1H, Pcp-H3a); 3.13-2.90 (m, 2H, Pcp-H4A,B); 2.80-2.62 (m, 2H, Pcp-H2A,B); 2.34 (s_{broad}, 1H, NH); 2.10-1.95 (m, 1H, Pcp-H3A); 1.63-1.47 (m, 1H, Pcp-H3B)

¹³C-NMR(CDCl₃):

δ(ppm)=152.6 (s, Pcp-C4a); 148.3 (d, Pcp-C8); 147.2 (d, Pcp-C6); 140.6 (s, Pcp-C8a); 119.9 (d, Pcp-C5); 67.0 (d, Pcp-C8b); 46.8 (t, Pcp-C2); 41.4 (d, Pcp-C3a); 38.5 (t, Pcp-C4); 35.6 (t, Pcp-C3)

### [1S-[1R*(R*),2(R*)]]-1,2,3,3a,4,8b-Hexahydro-N-(1-phenylethyl)-pyrrolo[3′,2′:4,5]-cyclopenta[1,2-c]-pyridino-1-carboxamide

10.15 g (63.4 mmol) of (±)-[3aα,8bα]-1,2,3,3a,4,8b-hexahydropyrrolo-[3′,2′:4,5]-cyclopenta-[1,2-c]pyridine in 230 ml of absol. acetone are admixed with 9.32 g (63.4 mmol) of (S)-(−)-α-methyl-benzenemethane-isocyanate in 20 ml of abs. acetone, stirred for 30 minutes at room temperature, and subsequently the solvent is removed.

The crude product is dissolved in 500 ml of acetic acid ethyl ester in boiling heat, stirred for 5 minutes over activated carbon, and filtered. The filtrate is admixed with seed crystals and allowed to crystallize for 3 hours at −20° C. The precipitated crystals are filtered off and digested twice with 5 ml each of ice-cold acetic acid ethyl ester.

Yield: 5.00 g of beige crystals (52% of theory)

TLC: EE:MeOH=8:1; 0.3

M.p.: 163-164° C. (acetic acid ethyl ester)

[α]_{D}²⁰: −218.3° (c=0.12/dichloromethane)

Microelementary analysis: HA35 C(%) H(%) N(%)
Summation formula: calculated: 73.38 6.94 13.51 C₁₉H₂₁N₃O*0.2H₂O found: 73.50 6.88 13.51

¹H-NMR(CDCl₃):
δ(ppm)=8.93 (s, 1H, Pcp-H8); 8.44 (d, 1H, Pcp-H6); 7.43-7.20 (m, 5H, Ph-H2,3,4,5,6); 7.13 (d, 1H, Pcp-H5); 5.46 (d, 1H, Pcp-H8b); 5.10 (dq, 1H, CH); 4.55 (d, 1H, NH,³J_{H, CH}=7.7 Hz); 3.42-3.26 (m, 2H, Pcp-H2A,B); 3.18-2.96 (m, 2H, Pcp-H3a,4A); 2.75 (d, 1H, Pcp-H4B); 2.31-2.11 (m, 1H, Pcp-H3A); 1.75-1.57 (m, 1H, Pcp-H3B); 1.40 (d, 3H, CH₃)

¹³C-NMR(CDCl₃):
δ(ppm)=156.1 (s, C═O); 150.4 (s, Pcp-C4a); 148.8 (d, Pcp-C8); 148.4 (d, Pcp-C6); 144.3 (s, Ph-C1); 140.1 (s, Pcp-C8a); 128.5 (d, 2C, Ph-C3,5); 127.0 (d, Ph-C4); 126.0 (d, 2C, Ph-C2,6); 120.2 (d, Pcp-C5); 65.0 (d, Pcp-C8b); 49.8 (d, CH); 45.8 (t, Pcp-C2); 40.9 (d, Pcp-C3a); 35.8 (t, Pcp-C4); 31.1 (t, Pcp-C3); 22.5 (g, CH₃)

### [1R-[1R*(R*),2(S*)]]-1,2,3,3a,4,8b-Hexahydro-N-(1-phenylethyl)-pyrrolo[3′,2′:4,5]-cyclopenta[1,2-c]-pyridino-1-carboxamide

The mother liquor of the above product is narrowed down to 300 ml, optionally dissolved in boiling heat, admixed with seed crystals and allowed to crystallize for 5 hours at −20° C. The precipitated crystals are filtered off and digested twice with 2 ml each of ice-cold acetic acid ethyl ester.

Yield: 1.20 g of colorless crystals (12% of theory)

TLC: EE:MeOH=8:1; 0.3

M.p.: 154-155° C. (acetic acid ethyl ester)

[α]_{D}²⁰: +185.6° (c=0.13/dichloromethane)

Microelementary analysis: HA36 C(%) H(%) N(%)
Summation formula: calculated: 72.96 6.96 13.43 C₁₉H₂₁N₃O*0.3H₂O found: 73.05 6.88 13.34

¹H-NMR(CDCl₃):
δ(ppm)=8.88 (s, 1H, Pcp-H8); 8.44 (d, 1H, Pcp-H6); 7.42-7.20 (m, 5H, Ph-H2,3,4,5,6); 7.13 (d, 1H, Pcp-H5); 5.40 (d, 1H, Pcp-H8b, ³J_{H,H3a}=7.4 Hz); 5.12 (dq, 1H, CH); 4.55 (d, 1H, NH); 3.45-3.25 (m, 2H, Pcp-H2A,B); 3.19-3.00 (m, 2H, Pcp-H3a,4A); 2.75 (d, 1H, Pcp-H4B); 2.31-2.15 (m, 1H, Pcp-H3A); 1.70-1.59 (m, 1H, Pcp-H3B); 1.57 (d, 3H, CH₃)

¹³C-NMR(CDCl₃):
δ(ppm)=156.3 (s, C═O); 150.5 (s, Pcp-C4a); 148.9 (d, Pcp-C8); 148.6 (d, Pcp-C6); 144.2 (s, Ph-C1); 140.2 (s, Pcp-C8a); 128.6 (d, 2C, Ph-C3,5); 127.2 (d, Ph-C4); 126.1 (d, 2C, Ph-C2,6); 120.5 (d, Pcp-C5); 65.1 (d, Pcp-C8b); 49.8 (d, CH); 46.0 (t, Pcp-C2); 41.1 (d, Pcp-C3a); 35.9 (t, Pcp-C4); 31.3 (t, Pcp-C3); 22.5 (q, CH₃)

### EXAMPLE 7

### (+)-[3aS-(3aα,8bα)]-1,2,3,3a,4,8b-Hexahydro-1-methyl-pyrrolo[3′,2′:4,5]-cyclopenta[1,2-c]pyridinodihydrochloride

650 mg (4.06 mmol) of [3aS-(3aα,8bα)]-1,2,3,3a,4,8b-hexahydropyrrolo[3′,2′:4,5]-cyclopenta[1,2-c]pyridine in 30 ml of abs. acetonitrile are admixed with 3.1 ml of 35% formaldehyde solution and subsequently in portions with 586 mg (9.33 mmol) of sodium cyanoborohydride. The reaction mixture is stirred for 30 minutes at room temperature.

No the pH is adjusted to 1 with 2N hydrochloric acid, and it is extracted twice with 30 ml of dichloromethane each. By the addition of 2N caustic soda solution, the aqueous phase is adjusted to pH=10, and it is extracted five times with 50 ml of dichloromethane each. The organic phase is dried over sodium sulfate/activated carbon, filtered, and the solvent is distilled off.

Yield: 650 mg of a yellow oil (92% of theory)

TLC: methylene chloride:methanol=8:1; R_{f}=0.25

With alcoholic acetic acid the product is converted into its dihydrochloride, it is crystallized under ethanol, filtered off and digested with acetone. The colorless crystals obtained are highly hygroscopical.

[α]_{D}²⁰: +13.2° (c=0.11/methanol)

Microelementary analysis: HA46 C(%) H(%) N(%)
Summation formula: calculated: 52.20 6.63 11.07 C₁₁H₁₆Cl₂N₂*0.33H₂O found: 52.25 6.71 10.88

¹H-NMR (D₂O):
δ(ppm)=9.08 (s, 1H, Pcp-H8); 8.76 (d, 1H, Pcp-H6); 8.05 (d, 1H, Pcp-H5); 5.50-5.26 (m, 1H, Pcp-H8b); 3.86-3.20 (m, 5H, Pcp-H2A,B,3a,4A,B); 3.15 (s, 3H, CH₃); 2.76-2.48 (m, 1H, Pcp-H3A); 2.10-1.81 (m, 1H, Pcp-H3B)

¹³C-NMR (D₂O):
δ(ppm)=168.8 (s, Pcp-C4a); 143.9 (d, Pcp-C8); 140.8 (d, Pcp-C6); 136.5 (s, Pcp-C8a); 126.3 (d, Pcp-C5); 76.5 (d, Pcp-C8b); 58.6 (t, Pcp-C2); 42.8 (q, CH₃); 42.0 (d, Pcp-C3a); 40.6 (t, Pcp-C4); 31.9 (t, Pcp-C3)

### EXAMPLE 8

### (−)-[3aR-(3aα,8bα)]-1,2,3,3a,4,8b-Hexahydro-1-methylpyrrolo[3′,2′:4,5]cyclopenta[1,2-c]pyridinodihydrochloride

from 580 mg (3.62 mmol) of [3aR-(3aα,8bα)]-1,2,3,3a,4,8b-hexahydropyrrolo[3′,2′:4,5]cyclopenta-[1,2-c]pyridine, 2.74 ml of 35% formaldehyde solution and 523 mg (8.33 mmol) of sodium cyanoborohydride analogous to Example 7 (90% of theory, colorless crystals).

[α]_{D}²⁰: −14.0° (c=0.14/methanol)

Microelementary analysis: HA47 C(%) H(%) N(%)
Summation formula: calculated: 52.42 6.61 11.11 C₁₁H₁₆Cl₂N₂*0.27H₂O found: 52.47 6.80 10.94

¹H-NMR(D₂O):

δ(ppm)=9.08 (s, 1H, Pcp-H8); 8.76 (d, 1H, Pcp-H6); 8.05 (d, 1H, Pcp-H5); 5.50-5.26 (m, 1H, Pcp-H8b); 3.86-3.20 (m, 5H, Pcp-H2A,B,3a,4A,B); 3.15 (s, 3H, CH₃); 2.76-2.48 (m, 1H, Pcp-H3A); 2.10-1.81 (m, 1H, Pcp-H3B)

¹³C-NMR (D₂O):
δ(ppm)=168.8 (s, Pcp-C4a); 143.8 (d, Pcp-C8); 140.8 (d, Pcp-C6); 136.7 (s, Pcp-C8a); 126.3 (d, Pcp-C5); 76.5 (d, Pcp-C8b); 58.7 (t, Pcp-C2); 42.8 (q, CH₃); 42.1 (d, Pcp-C3a); 40.6 (t, Pcp-C4); 31.9 (t, Pcp-C3)

### EXAMPLE 9

### Radioligand Assay

The [³H] cytisin bond to the α4β2-subtype in rat brain membranes was determined by a modified method of Pabreza et al. (Pabreza, L. A., Dhawan, S., Kellar, K. J., Mol. Pharmacol. 1991, 39, 9-12):

Membrane-enriched fractions of rat brain without cerebellum (ABS Inc. Wilmington, Del.) were slowly thawed at 4° C., washed, and re-suspended in 30 parts of BSS-Tris buffer (120 mM NaCl, 5 mM KCl, 2 mM CaCl₂, 2 mM MgCl₂ and 50 mM Tris-Cl, pH 7.4, 4° C.). Dilutions of the test compound (10⁻⁵ to 10⁻¹¹M) which contain 100-200 μg of protein and 0.75 nM [³H] cytisin (30 Ci/mmol; Perkin Elmer NEN, Boston, Mass.) were incubated in a final volume of 500 μl for 75 minutes at 4° C. (2 samples each). The non-specific binding was determined with 10 μM (−)nicotine. The incubation was determined by vacuum filtration through a Whatman GF/C filter which previously had been humidified with 0.5% polyethylene imine. Bound radioactivity was collected on Millipore Multiscreen plates FB with a Packard Cell Harvester, and determined with a Packard Topcount Microplate Beta Counter. IC₅₀ values were determined by non-linear regression, and from this the Kᵢ values by means of the Cheng-Prusoff equation, wherein Kᵢ=IC₅₀/1+[ligand]/K_{ID}. The mean Kᵢ values were obtained from at least three individual determinations.

Kᵢ Values [nM] of the Enantiomer Pairs

| | | | |
|---|---|---|---|
| Example 1 | 2430 | Example 2 | >1 mM |
| Example 3 | 199 | Example 4 | >1 mM |
| Example 5 | 1110 | Example 6 | 15.2 |
| Example 7 | 1670 | Example 8 | 27.0 |

1.-14. (canceled)

15. A compound further defined as an enantiomerically pure [3aα,8bα]-1,2,3,3a,4,8b-hexahydropyrrolocyclopentapyridine derivative or pharmaceutically usable salt thereof of formula: 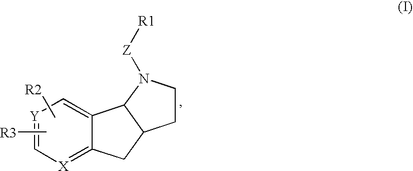
wherein:
Z is a single bond or CH₂;
R1 is a hydrogen or a straight-chain or branched lower alkyl residue;
R2 and R3 are independently a hydrogen, a straight-chain or branched lower alkyl residue, a lower alkoxy, a lower alkylthio, or a halogen; and
X and Y alternately are CH or N.

16. The compound of claim 15, wherein at least one of R1, R2, and/or R3 is unsaturated and/or perfluorated.

17. The compound of claim 16, wherein at least one of R1, R2, and/or R3 are hydrogen.

18. The compound of claim 15, further defined as (+)-[3aα,8bα]-1,2,3,3a,4,8b-Hexahydropyrrolo-[2′,3′:3,4]cyclopenta[1,2-b]pyridine dihydrochloride.

19. The compound of claim 15, further defined as (+[3aα,8bα]-1,2,3,3a,4,8b-Hexahydro-1-methyl-pyrrolo-[2′,3′:3,4]cyclopenta[1,2-b]pyridine dihydrochloride.

20. The compound of claim 15, further defined as (−)-[3aR-(3aα,8bα)]-1,2,3,3a,4,8b-Hexahydropyrrolo-[3′,2′:4,5]cyclopenta[1,2-c]pyridine dihydrochloride.

21. The compound of claim 15, further defined as (−)-[3aR-(3aα,8bα)]-1,2,3,3a,4,8b-Hexahydro-1-methyl-pyrrolo-[3′,2′:4,5]cyclopenta[1,2-c]pyridine dihydrochloride.

22. A method for producing a compound of formula (I) comprising:
reductively converting into a compound of formula (I) a compound of the general formula (II) 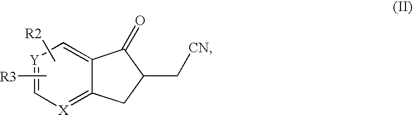
wherein:
Z is a single bond;
R1 is hydrogen;
R2 and R3 are independently a hydrogen, a straight-chain or branched lower alkyl residue, a lower alkoxy, a lower alkylthio, or a halogen; and
X and Y alternately are CH or N;
to obtain a diastereomer mixture;
recovering a less readily soluble diastereomer from the diastereomer mixture by crystallization to obtain a diastereomerically pure compound of the general formula (III): 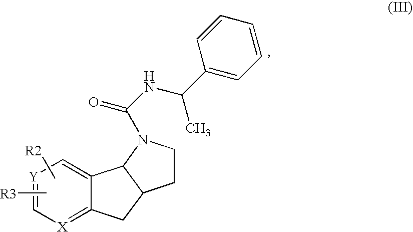
and
cleaving the diastereomerically pure compound of the general formula (III) under suitable conditions to give the enantiomerically pure compound of the general formula (I).

23. The method of claim 22, further comprising reacting the compound of formula (III) with enantiomerically pure 1-phenylethylisocyanate.

24. The method of claim 22, further comprising reacting the enantiomerically pure compound of the general formula (I), wherein Z is a single bond and R1 is a hydrogen, under alkylating conditions to compounds of the general formula (I), wherein Z is CH2.

25. The method of claim 22, further comprising converting the compound of the general formula (I) into a pharmaceutically usable salt.

26. A pharmaceutical preparation comprising a compound of claim 15 and at least one galenic auxiliary and/or carrier substance.

27. The pharmaceutical preparation of claim 26, further defined as comprising an additional therapeutically valuable compound.

28. A method comprising obtaining a compound of claim and administering it to a subject.

29. The method of claim 28, wherein the subject has a disease of the central nervous system.

30. The method of claim 28, wherein the disease of the central nervous system is dementia caused by old age, Alzheimer's Disease, Parkinson Disease, Tourett's Syndrome, dyskinesia anxiety, depression, panic, psychosis, bulimia, or anorexia.

31. The method of claim 28, wherein the subject is in need of an analgesic, nociceptive agent, and/or neuroprotective agent.

32. The method of claim 28, further defined as a method of improving perception and/or attention in the subject.

33. The method of claim 28, wherein the subject is in smoke substitution therapy.
